(19) United States
(12) Patent Application Publication
(10) Pub. No.: US 2026/0306193 A1
(43) Pub. Date: Oct. 8, 2026

(54) ARTICULATION MECHANISM CONFIGURATIONS AND DRIVE SYSTEMS, AND RELATED DEVICES AND METHODS

(71) Applicant: INTUITIVE SURGICAL OPERATIONS, INC., Sunnyvale, CA (US)

(72) Inventors: Andrew C. Waterbury, New Canaan, CT (US); Rumen Deyanov, Fremont, CA (US); Bram Gilbert Antoon Lambrecht, Redwood City, CA (US)

(73) Assignee: INTUITIVE SURGICAL OPERATIONS, INC., Sunnyvale, CA (US)

(21) Appl. No.: 19/479,079

(22) PCT Filed: Apr. 25, 2024
(86) PCT No.: PCT/US2024/026139

§ 371 Date: Oct. 27, 2025

Related U.S. Application Data
(60) Provisional application No. 63/498,898, filed on Apr. 28, 2023.

(51) Int. Cl. A61B 34/30; A61B 34/00

(52) U.S. Cl. CPC A61B 34/30; A61B 34/71; A61B 2034/305

(57) ABSTRACT

A system for actuating an articulation mechanism can include a link extending along a longitudinal dimension from a proximal end portion to a distal end portion of the link. An articulation mechanism coupled to the distal end portion of the link is articulatable in pitch and yaw relative to the link. A first motor can be housed at least partially in the link and operably coupled to transmit force to actuate the articulation mechanism to articulate in pitch relative to the link, and a second motor can be housed at least partially in the link and operably coupled to transmit force to actuate the articulation mechanism to articulate in yaw relative to the link. The first motor and the second motor can be arranged consecutively along the longitudinal dimension such that the first motor is closer to the proximal end portion of the link than the second motor.

### CROSS-REFERENCE TO RELATED APPLICATIONS

This application claims the benefit of U.S. Provisional Application No. 63/498,898, filed Apr. 28, 2023, the entirety of which is incorporated by reference herein.

### FIELD

Aspects of this disclosure relate generally to articulation mechanisms used to provide movement of a kinematic chain in multiple degrees of freedom. For example, aspects of the disclosure relate to drive systems for an articulation mechanism of a manipulator of a manipulator system, such as a medical manipulator system, that can articulate in pitch and yaw degrees of freedom. Related devices and methods also are disclosed.

### INTRODUCTION

Various mechanical kinematic chains employ various join mechanisms, to provide one or more of various degrees of freedom of movement to the various mechanical links of the chain. By way of example, such joint mechanisms can provide translational movement of a link, roll movement of a link, and articulation (pivoting) movement of a link (e.g., in pitch and/or yaw). Some applications require multiple degrees of freedom movements with relatively small-sized kinematic chains, and thus relatively limited space for joint mechanisms to achieve all of the desired motion of the kinematic chain. Further, in some applications, it is desirable to actuate movement of such mechanisms through a drive device (as opposed to having the joint mechanism be passively moved or manipulated manually), such as, for example, an electric motor, or a hydraulic or pneumatic drive device. Using drive devices to actuate movement of joint mechanisms in kinematic chains can present challenges in some applications, such as the mass (weight) of the drive device impacting the overall size and/or movement of the kinematic chain, and heat that may be associated with the drive device (e.g., particularly in the case of an electric motor).

Computer-assisted manipulator systems (“manipulator systems”), sometimes referred to as robotically-assisted systems, robotic systems, or teleoperated systems may include one or more manipulators that can be operated with the assistance of an electronic controller (e.g., computer) to move and control functions of one or more instruments when coupled to the manipulators. A manipulator generally includes a kinematic chain of mechanical links connected by joint mechanisms, some of which may be actuated by drive devices. An instrument is removably couplable to (or permanently coupled to) one of the links, typically a distal link of the plural links. The joint mechanisms are operable to cause the links to move (i.e., rotate, roll, and/or translate) relative to one another, imparting various degrees of freedom to the manipulator to enable the manipulator to move the instrument relative to a worksite.

The manipulators of a manipulator system can be used to transmit a variety of forces and torques to the instruments to perform various procedures, such as medical procedures or non-medical procedures (e.g., industrial procedures). In such cases, the link to which the instrument is couplable or coupled (e.g., via an instrument carriage) includes drive outputs to interface with and mechanically transfer driving forces to corresponding drive inputs of the instrument to control degrees of freedom of motion and/or other functions of the instrument. In the case of medical procedures, the manipulators can be attached to a manipulator support structure (e.g., a patient side cart) that is separate from a support structure that supports a body (e.g., patient or workpiece), or they may be attached directly to the support structure that supports the body (e.g., to an operating table), the latter being referred to herein as “table-mounted.”

A need exists to address various challenges of utilizing joint mechanisms, including articulation mechanisms, in kinematic chains that can provide for a number of degrees of freedom of motion in relatively compact space arrangements. Moreover, a need exists to address various challenges associated with actuation of such joint mechanisms. Further, a need exists to address one or more of these challenges in the context of a manipulator of a computer-assisted manipulator system, including, for example, for use in medical procedures.

### SUMMARY

Various embodiments of the present disclosure can solve one or more of the above-mentioned problems and/or can demonstrate one or more of the above-mentioned desirable features. Other features and/or advantages will become apparent from the description that follows.

Various embodiments of the present disclosure contemplate a system for actuating an articulation mechanism. The system can include a link extending along a longitudinal dimension from a proximal end portion of the link to a distal end portion of the link. The link can include an articulation mechanism coupled to the distal end portion of the link, the articulation mechanism articulatable in pitch and yaw relative to the link. The system can further includes a first motor housed at least partially in the link and operably coupled to transmit force to actuate the articulation mechanism to articulate in pitch relative to the link, and a second motor housed at least partially in the link and operably coupled to transmit force to actuate the articulation mechanism to articulate in yaw relative to the link. The first motor and the second motor can be arranged consecutively along the longitudinal dimension such that the first motor is closer to the proximal end portion of the link than the second motor.

Implementations can also include one or more of the following aspects, alone or in combination with one or more of the other aspects. The second motor can be distal to the first motor. The first motor can be located at a proximal end portion of the link. The articulation mechanism can include a first joint defining a pitch axis and a second joint defining a yaw axis. The first joint and second joint can be offset from each other along the longitudinal dimension. The first joint can be proximal the second joint. The first joint can include a first cable-and-pulley assembly operably coupled to the first motor, and the second joint can include a second cable-and-pulley assembly operably coupled to the second motor. The first cable-and-pulley assembly can include a first pair of cables respectively operably coupled to a first pulley mechanism having a rotation axis along the pitch axis, the first pair of cables configured to be oppositely paid in and paid out in response to rotation of the first motor. The second cable-and-pulley assembly can include a second pair of cables respectively operably coupled to a second pulley mechanism having a rotation axes along the yaw axis, the second pair of cables configured to be oppositely paid in and paid out in response to rotation of the second motor. The first pair of cables can extend past and laterally of the second motor between the first motor to the first pulley mechanism. The second pair of cables can wrap on the first pulley mechanism in opposite directions.

The system can further include a first capstan operably coupled to the first motor, the first pair of cables at least partially wrapping around the first capstan in opposite directions, and a second capstan operably coupled to the second motor, the second pair of cables at least partially wrapping around the second capstan in opposite directions. The first motor and the second motor can be configured to actuate the articulation mechanism independently in pitch and yaw.

The system can further include one or more braking mechanisms configured to apply a braking force to brake one or both of the first motor and the second motor. The articulation mechanism can be configured to be locked in pitch articulation in response to the braking mechanism applying the braking force to the first motor. The articulation mechanism can configured to be locked in yaw articulation in response to the braking mechanism applying the braking force to the first motor.

The link can be a link of a manipulator arm of a teleoperated manipulator system, and the manipulator arm can include a manipulator drive interface configured to transmit actuation force to a medical instrument mounted to the manipulator drive interface. The manipulator arm can further include a spar link coupled to the link via the articulation mechanism, the manipulator interface coupled to the spar link. The link can be a distal link of the manipulator arm and be rotatably coupled to a proximal link of the manipulator arm. The distal link can be configured to roll about a longitudinal axis of the distal link relative to the proximal link. The system can include a third motor proximal the first motor and operably coupled to transmit force to actuate the distal link to roll about the longitudinal axis.

In various embodiments, the present disclosure contemplates a system for actuating an articulation mechanism. The system can include a link extending along a longitudinal dimension from a proximal end portion of the link to a distal end portion of the link; an articulation mechanism coupled to the distal end portion of the link, the articulation mechanism articulatable in pitch and yaw relative to the link. The articulation mechanism can comprise a first cable-and-pulley assembly comprising a first pair of cables respectively operably coupled to a first pulley mechanism having a rotation axis defining a pitch articulation axis of the articulation mechanism, the first pair of cables configured to be oppositely paid in and paid out relative to rotation of the first pulley mechanism. The articulation mechanism can further comprise a second cable-and-pulley assembly comprising a second pair of cables respectively operably coupled to a second pulley mechanism having a rotation axis defining a yaw articulation axis of the articulation mechanism, the second pair of cables configured to be oppositely paid in and paid out relative to rotation of the second pulley mechanism. The second pair of cables can wrap on the first pulley mechanism in opposite directions.

The system can further include one or more of the following aspects. The pitch articulation axis and the yaw articulation axis can be offset from each other. The pitch articulation axis can be proximal to the yaw articulation axis.

The system can further include a first capstan, the first pair of cables operably coupled to the first capstan and configured to be respectively paid out or paid in in response to rotation of the first capstan. The system can further include a second capstan, the second pair of cables operably coupled to the second capstan and configured to be respectively paid out or paid in in response to rotation of the second capstan. A first cable of the first pair of cables can wrap at least partially around the first idler pulley, and a second cable of the first pair of cables can wrap at least partially around the second idler pulley. The first idler pulley rotation axis and the second idler pulley rotation axis can be offset relative to the pitch rotation axis in a direction perpendicular to the longitudinal dimension. The first idler pulley rotation axis and the second idler pulley rotation axis can be offset in opposite directions from each other relative to the pitch rotation axis.

The system can further include a flux transmission conduit following a path extending along the link through the articulation mechanism, the flux transmission conduit following a clockspring path around the pitch articulation axis and U-shaped path through the yaw articulation axis, wherein the U-shaped path can extend transverse to the yaw articulation axis.

The system can further include a first drive device operably coupled to the first pair of cables and configured to transmit force to pay in and pay out the first pair of cables; and a second drive device to the first pair of cables and configured to transmit force to pay in and pay out the first pair of cables. The first drive device and second drive device can be housed in the link. The first drive device can be proximal to the second drive device along the length dimension of the link.

In various embodiments, the present disclosure contemplates a capstan. The capstan can include a pair of annular plates removably attached together and rotatable about an axis of rotation. A groove can be defined between the pair of plates along a peripheral surface between the pair of annular plates, the groove configured to receive and at least partially wrap a cable around the capstan. One or both of the pairs of plates can include an inner surface defining a cable routing feature configured to capture and route a cable in the attached state of the pair of plates. The cable routing feature can extend from a location between the axis of rotation and the peripheral surface to a location along the groove at the peripheral surface.

The capstan can further include one or more of the following aspects. The cable routing feature can include a pocket configured to capture a crimp on an end portion of a cable wrapped on the capstan. The pair of plates can be a first pair of plates and the capstan can further include one or more additional pairs of plates, each pair of plates configured to wrap a separate respective cable on the capstan. Each pair of plates can be configured to be operably coupled to a common motor shaft and individually rotatable relative to the shaft to individually modify tension in respective cables. Each pair of plates can include an engagement feature configured to removably engage with a complementary engagement feature of a tightening tool.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1 is a schematic view of a kinematic chain including a mechanical link, an articulation mechanism at a distal end portion of the mechanical link, an instrument supported by the articulation mechanism, and a drive system operably coupled to the articulation mechanism in accordance with one embodiment;

FIG. 2 is a schematic view of an embodiment of articulation mechanism and cable-pulley drive system for actuating the articulation mechanism;

FIG. 3 is a perspective view of an embodiment of a distal link of a manipulator supporting a spar carrying an instrument carriage and a cannula mount, and showing an instrument coupled to the carriage and a cannula couped to the cannula mount;

FIG. 4 is a perspective view of an embodiment of a manipulator system which can include one or more manipulators including the components of FIG. 3;

FIG. 5. is a side, perspective view of an embodiment of a distal kinematic chain of a manipulator housing a drive system for an articulation mechanism of the distal kinematic chain;

FIG. 6 is a top perspective view of the distal kinematic chain and drive system of FIG. 5;

FIG. 7 is a partial side view of the distal kinematic chain and drive system of FIG. 5;

FIG. 8 is a partial side view, opposite the view of FIG. 7;

FIG. 9 is another partial side view of the distal kinematic chain and drive system of FIG. 5;

FIG. 10 is a partial side view, opposite the view of FIG. 9;

FIG. 11 is a plan view of an embodiment of a capstan module with a cable wrapped on the module;

FIGS. 12A and 12B are perspective views of embodiments of annular plates of a capstan module;

FIG. 13 is a perspective view showing a rack and pinion tensioning tool engaged with an in situ capstan including capstan modules in accordance with an embodiment; and

FIG. 14 is a partial sectional view of a distal end portion of a kinematic chain illustrating a flux conduit routing path in accordance with an embodiment.

FIG. 15 is a isolated, partial sectional view of a shaft that forms part of the flux conduit routing path of FIG. 14.

FIG. 16 is an isolated perspective view of a spar coupling member of FIGS. 14 and 15.

### DETAILED DESCRIPTION

In various applications, it may be desirable to provide articulation mechanisms that provide multiple degrees of freedom located relatively closely together along a kinematic chain. This may allow for better control of the ultimate desired motion.

For example, in the context of a medical manipulator, providing various degrees of freedom of movement (“DOFs”) at a distal link of the kinematic chain of the manipulator, so as to allow a precise and varied orientation of an instrument coupled at the distal link, is desirable to help minimize the risk of the more proximal links of the manipulator undesirably contacting objects during motion thereof. Providing various DOFs is further desirable due to the otherwise limited space in which the more proximal links have to move to achieve desired orientations of the instrument, particularly considering their overall size. Moreover, arranging the various joint structures providing the various DOFs close to each other and to a target remote center of motion of the instrument coupled to the distal link (e.g., proximate the location of insertion of the instrument into the body (of the workpiece or patient) can assist in limiting the volume swept out by the manipulator to achieve the desired instrument motion. In other words, the overall motion of the kinematic chain making up the manipulator arm can be concentrated near the target remote center, where it sweeps out less volume during its motion and where it also may be easier to monitor during a procedure. Various embodiments described herein can achieve articulation mechanism arrangements that include multiple joint structures, providing differing DOFs, located relatively close together and near a target remote center of motion.

As used herein, in the context of a medical manipulator that is configured to support an instrument to orient the instrument during a procedure, an articulation mechanism located at a distal end portion of a distal link of the manipulator kinematic chain structure is also referred to as a wrist mechanism, with the overall manipulator structure being considered a manipulator arm. References to a wrist mechanism herein may be used interchangeably with articulation mechanism herein, for example, when referring to a kinematic chain of a manipulator arm with the articulation mechanism located at a distal end portion of the arm so as to be able to orient an instrument held at a distal end of the arm. However, the disclosed articulation mechanisms are not limited to having a specific configuration as a wrist from human anatomy and further could be located in other portions of kinematic chains depending on the desired application.

It also may be desirable to provide such joint structures of an articulation mechanism that are actuated by drive devices, such as motors or pneumatic/hydraulic drive devices. Various embodiments herein address challenges that may arise when using such drive devices for the actuation of joints, including challenges with space constraints, movement control, and/or heat that may be undesirable if such drive devices were located in close proximity of the joints. For example, in the context of a medical manipulator with an articulation mechanism comprising one or more joint structures located at a distal end portion of the distal link in the proximity of where the instrument is coupled to the link, heat associated with a drive device, such as an electric motor, may be undesirable due to its proximity to an insertion location of the instrument in a body. Moreover, the size and mass associated with such drive devices can pose challenges relating to the overall space limitations and may have an effect on the dynamics of motion that exist when joints are located at a distal link, particularly when the joints are located close to the location where the instrument is coupled.

Various embodiments address these challenges by utilizing drive systems that rely on drive devices located at positions relatively distant from the location of the joint structures. For example, for the distal link of a manipulator supporting an instrument in a computer-assisted manipulator system, drive devices may be located at proximal portions of the distal link and connected via actuation members (e.g., cables or other similar tension members), extending between the drive devices and the joint mechanisms. As used herein, the term “cable” is used to connote any of a variety of tension members that are configured to be flexible and permit wrapping around an object (such as, for example, a pulley, capstan, shaft, or the like) but able to transmit a force when tensioned. Cables as used herein can thus include, but are not limited to, wires, ropes, filaments, strips, bands, and similar structures. Cable as used herein is not limited to a structure that is braided or includes multiple stands, although such structures are encompassed by the term. Various embodiments of drive systems utilize drive devices and actuation member routing arrangements to further address space constraints in the overall distal link and to permit the relatively close proximity of multiple joint structures used to form an articulation mechanism, such as a wrist mechanism at a distal end portion of an manipulator or other kinematic arm structure.

The present disclosure further contemplates various embodiments to address challenges associated with tensioning actuation members and/or wear issues associated with utilizing a relatively remotely located drive devices in conjunction with tensioned actuation members to transmit drive forces to actuate motion of the joint mechanisms. Further, embodiments that can permit routing of other flexible conduits (e.g. cable and/or other lines) for transmitting electrical energy or other flux supply (e.g., fluids, suction, light, etc.) through the link and joint mechanisms are contemplated.

Turning now to the Figures, some non-limiting embodiments of articulation mechanisms, including associated joint structures and drive systems for the same, and various related components, device, and methods are described.

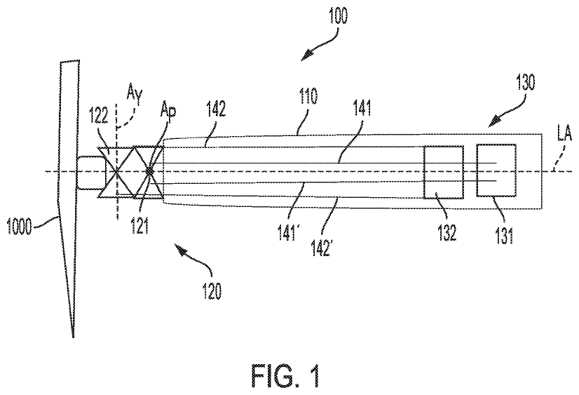

FIG. 1 schematically depicts an embodiment of a kinematic chain 100 including a mechanical link 110 having a longitudinal axis L_{A} that is configured to support an instrument 1000 at a distal end portion of the link 100, with a distal direction being toward the instrument supporting end portion of the link 100 and a proximal direction being in the opposite direction as reflected by the arrows in FIG. 1. The kinematic chain 100 further includes an articulation mechanism 120 including two joint structures 121, 122 configured to respectively articulate around differing axes, which in some embodiments can be orthogonal to each other through at least some of the range of articulation of each joint. In the embodiment of FIG. 1, the joint structure 121 is configured to articulate about an axis A_{P} to provide pitch articulation relative to the link 110 and the joint structure 122 is configured to articulate about an axis A_{Y} to provide yaw articulation relative to the link 110. Thus, the articulation mechanism 120 can thus be considered as a wrist mechanism 120 and provide various orientations of the instrument 1000 by articulation of the articulation mechanism 120. The position of the wrist mechanism 120 is also located close to a remote center of motion, which can be constrained either mechanically or via software in a computer-assisted embodiment, of the instrument 1000.

The embodiment of FIG. 1 further includes a drive system 130 that includes drive devices 131, 132 located remotely from the joint structures 121, 122 at a proximal end portion of the link 110. As discussed above, locating drive devices more proximally in the link 110 can be desirable to move the weight of the drive devices 131, 132 away from the instrument 1000 and remote center of motion, thereby providing more control over dynamics associated with movement of the wrist mechanism 120 and over other motions, such as, for example, in embodiments involving other degrees of freedom of motion of an instrument and link supporting the instrument as described further below. Further, the location of drive devices 131, 132 away from the wrist mechanism 120 and distal end portion of the link 110 can be beneficial for drive devices, such as motors for example, that produce heat. In various applications, such as in a medical system, heat can be undesirable when located near the instrument 1000 performing a procedure, such as a medical procedure.

Actuation members can be used to transmit forces from the drive devices 131, 132 to the joint structures 121, 122 of the wrist mechanism 120. In various embodiments, actuation members in the form of tension members, such as cables or similar members, can be used. As shown, a pair of cables 141, 141′ can be coupled between the drive device 131 and the joint structure 121 and another pair of cables 142, 142′ can be coupled between the drive device 132 and the joint structure 122. The drive device 131 can transmit force to respectively pay in and pay out the pairs of cables 141 and 141′ to articulate the joint structure 121 in opposite directions about the axis A_{P}. The drive device 132 can transmit force to respectively pay in and pay out the pairs of cables 142 and 142′ to articulate the joint structure 122 in opposite directions about the axis A_{Y}. In other words, paying in a cable causes tension in the cable tending to pull it in the proximal direction to articulate the corresponding joint in response, the other cable of the pair is simultaneously paid out to allow the articulation to occur.

As shown in FIG. 1, in various embodiments to address space constraints that can exist in the mechanical link 110, the drive devices 131, 132 are located in series along a longitudinal dimension of the link 110 (e.g., a dimension extending parallel to the longitudinal axis L_{A}). In various embodiments, the drive devices 131, 132 are electric motors, but the present disclosure contemplates other types of drive devices, such as, for example, pneumatic or hydraulic drive devices. In some embodiments, the more proximally located drive device, e.g., 131 in the embodiment of FIG. 1, can be a larger drive device than the more distally located drive device, e.g., 132 in the embodiment of FIG. 1. Such a configuration can be desirable in cases in which the joint structure to which it is connected, e.g., joint structure 121 in the embodiment of FIG. 1, requires a larger range of motion and/or forces to be transmitted thereto for articulation.

Those having ordinary skill in the art would appreciate that the locations of the joint structures 121, 122 of wrist mechanism 120 can be reversed and so can the drive devices 131, 132 which operate the joint structures 121, 122. Moreover, while two cables 141, 141′ and 142, 142′ are shown in the embodiment of FIG. 1 to transmit the drive forces from each drive device 131, 132 to the respective joint structures 121, 122, it is contemplated that additional cables can be used to provide redundant force transmission. For example, in various embodiments additional pairs of cables can be used to assist in sharing the load associated with transmitting drive force from a drive device to an operably coupled joint structure.

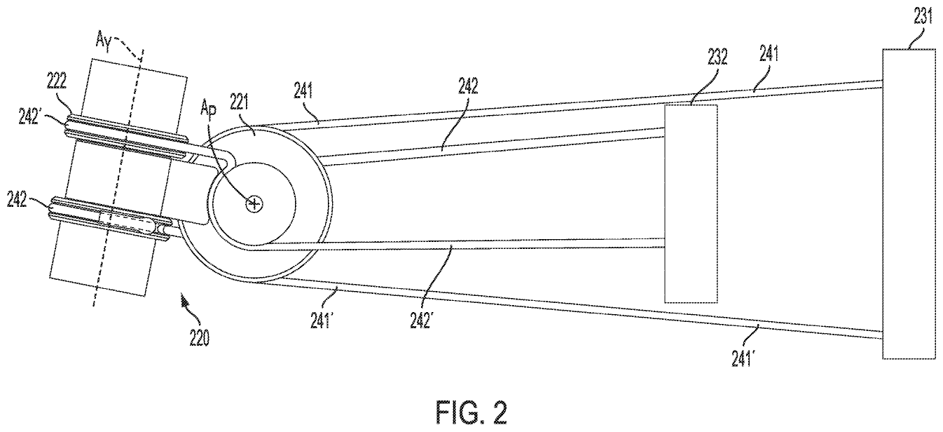

In various embodiments, drive systems employing cable-pulley and cable routing arrangements can be employed to further conserve space, while maintaining desirable joint stiffness and other desirable dynamics, when utilizing more proximally located drive devices for actuation of joint structures of a wrist mechanism at a distal end portion of a mechanical link. FIG. 2 schematically illustrates an embodiment of a cable-driven wrist mechanism utilizing an arrangement of cables and pulleys to actuate a wrist mechanism including a pitch articulatable joint structure and a yaw articulatable joint structure. The arrangement permits a wrist mechanism and drive system of relatively small dimensions to be employed, while providing sufficient joint stiffnesses and overall relatively large range of motions of the joint structures. In the arrangement of FIG. 2, a wrist mechanism 220 is illustrated that can be one embodiment of the wrist mechanism 120 and operably coupled cables 141, 141′, 142, 142′. The wrist mechanism 220 includes joint structures 221, 222 that include a pulley mechanism rotatable about axis A_{P} and a pulley mechanism rotatable about an axis A_{Y}, wherein the axes A_{P} and A_{Y} are orthogonal to each other and thus can be used as joint structures articulatable in pitch and yaw, respectively, such as for joint structures 121, 122 in the embodiment of FIG. 1.

As in the embodiment of FIG. 1, a pair of cables 241, 241′ operably couples a drive device 231 and the pulley mechanism of joint structure 221 and a pair of cables 242, 242′ operably couples the drive device 232 and the pulley mechanism of joint structure 222. The pair of cables 241, 241′ wrap in opposite directions about the pulley mechanism 221 of joint structure 221 and the pair of cables 242, 242′ wrap in opposite directions around the pulley mechanism 222. This enables the pair of cables 241, 241′ to pay in and pay out oppositely in response to rotation of the pulley mechanism of joint structure 221 and likewise for the pair of cables 242, 242′ to pay in and pay out oppositely in response to rotation of the pulley mechanism of joint structure 222.

Because of the close proximity of the pulley mechanisms of the joint structures 221, 222 (and thus the pitch and yaw axes A_{P}, A_{Y} of articulation of the wrist mechanism 220) the cables 242, 242′ extending from the pulley mechanism of joint structure 222 passes the pulley mechanism of joint structure 221 to operably couple to the drive device 232. By routing the cables 242, 242′ to the pulley mechanism of joint structure 221, again in oppositely wrapped directions relative to pulley mechanism, as well as coupling the cables 242, 242′ to the pulley mechanism of joint structure 222, length conservation of the cables 242, 242′ can be maintained when joint structure 221 is actuated by the drive mechanism 231 by relative paying in and paying out of the cables 241, 241′.

While the coupling and routing of the cables 242, 242′ around the pulley mechanism of joint structure 221 can assist in making the overall wrist mechanism 220 and drive components for the wrist mechanism 220 relatively compact, the coupling results in the need to drive both joint structures 221, 222 via drive devices 231, 232 to orient the wrist mechanism 220 in the pitch articulation only. In other words, as the pulley mechanism of joint structure 221 is driven to rotate about axis A_{P} to pay in/pay out the cables 241, 241′, the cables 242, 242′ that also wrap in opposite directions around the pulley mechanism of joint structure 221 will cause the joint structure 222 to rotate responsively about axis A_{Y}.

Accordingly, if the rotation of the pulley mechanism 222 is not desired when actuating the rotation of the pulley mechanism 221 is being driven by drive device 231, the drive device 232 can be driven to actuate the rotation of pulley mechanism 222 in the opposite direction as would otherwise occur in response to the actuation of pulley mechanism 221. In other words, drive device 232 can be driven to counteract the responsive rotation of the pulley mechanism 222 that would occur based on the drive device 231 actuating pulley mechanism 221. Notably, however, actuation of the pulley mechanism 222 via the cables 242, 242′ and drive device 232 does not affect the actuation or rotation of pulley mechanism 221 because the cables 242, 242′ pass around the pulley mechanism 221 without being fixedly coupled thereto.

As a further result of the above-described coupling of the cables 242, 242′ to the pulley mechanism of joint structure 221, a safety mechanical braking mechanism that is applied to drive device 232 can be used to effectively brake the actuation of both pulley mechanisms of joint structures 221, 222, thus eliminating the need for a separate safety braking mechanism for drive device 231, which can further reduce the overall dimensions and space requirements of the kinematic chain and drive system. In other words, a braking mechanism applied to the drive device associated with the joint structure that provides the independent DOF (not coupled to the other joint structure), can act as the braking mechanism for the other, dependent DOF (coupled) joint structure.

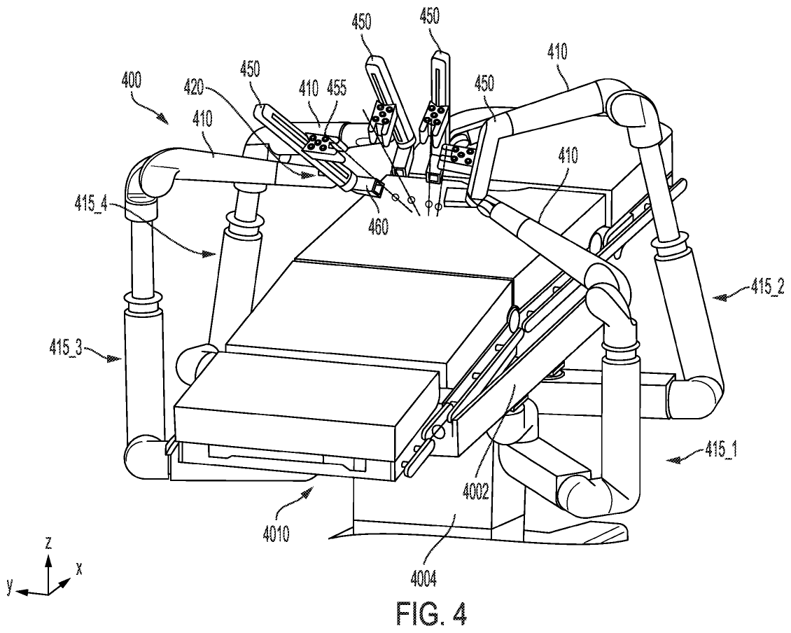

A kinematic chain including a wrist mechanism at a distal end portion of a mechanical link and drive system in accordance with any of the embodiments discussed above with reference to FIG. 1 and 2 can be implemented as part of a manipulator for orienting and transmitting drive forces to an instrument coupled to the manipulator in a computer-assisted system that employs robotic technology. Examples of such manipulator systems can include medical manipulator systems such as those used in robotic surgery, such as but not limited to, the da Vinci® Surgical Systems commercialized by Intuitive Surgical, Inc., of Sunnyvale, California. Other manipulator systems, including so-called table-mounted manipulator systems, are also contemplated, with one embodiment of a table-mounted manipulator system being depicted in FIG. 4 for example and further described below.

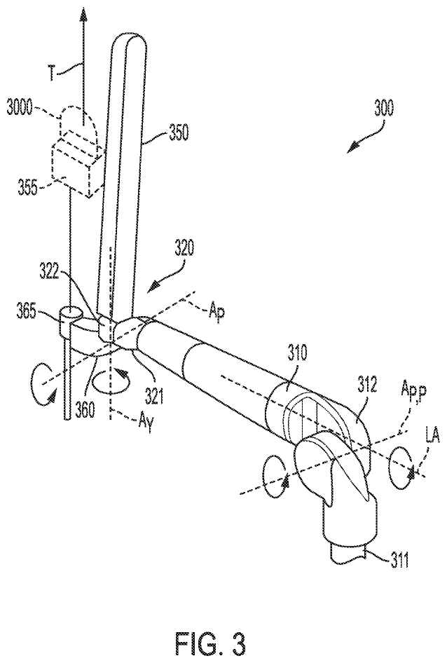

FIG. 3 illustrates one embodiment of a distal kinematic chain 300 of that can be part of a manipulator for transmitting drive forces to an instrument, such as a manipulator of a computer-assisted manipulator system for performing medical procedures using a medica instrument. The distal kinematic chain 300 of the manipulator includes a distal link 310, a wrist mechanism 320 at a distal end portion of the link 310, with the wrist mechanism 320 supporting a spar link 350 (hereinafter “spar”) and a cannula mount 360. The spar 350 supports a carriage 355 that is translatable along the spar 350 in directions T shown by the double arrows in FIG. 3. A medical instrument 3000 (shown schematically as a dashed line) can be coupled (e.g., removably or permanently) to the carriage 355 and inserted through a cannula 365 held by the cannula mount 360. The carriage 355 incudes a manipulator drive interface that operably couples to one or more drive inputs of the instrument, as those of ordinary skill in the art are familiar, to transmit various drive forces to actuate motion and other functionality of portions of the instrument 3000 in a coupled state of the instrument 3000 to the carriage 355.

As shown in FIG. 3, the distal kinematic chain 300 can provide five of six degrees of freedom of movement that are desirable to ultimately orient the instrument 3000. These five degrees of freedom include rotation of the distal link 310 at proximal joint 312 about an axis A_{P,P}, roll of the distal link 310 about the longitudinal axis L_{A} of the link 310 (which can be provided by an additional motor in the distal link), the pitch and yaw articulation about respective axes A_{P} and A_{Y} at the respective joint structures 321, 322 of wrist mechanism 320, and linear translation along directions T. As discussed above, locating various ones of these degrees of freedom closer to the instrument 3000 coupled to the carriage 355, and thus a remote center of motion of the instrument (whether defined and controlled via software or otherwise), can be desirable to provide better control over orientation of the instrument 3000 while sweeping less volume with portions of the manipulator proximal to the instrument 3000, such as link 310 and other more proximal links, as described below.

The link 310 can be a distal link of an overall arm of the manipulator and thus other links more proximal to link 310 can be included as part of the overall manipulator arm, with the other one or more proximal links being coupled via various joint structures so as to provide desired motion. FIG. 3 illustrates a portion of one such proximal link 311 to which the distal link 310 is coupled shown in FIG. 3. An embodiment of manipulators with additional proximal links are also depicted as part of the table-mounted manipulator system shown in the embodiment of FIG. 4, such manipulators 415 being one embodiment of a manipulator incorporating a distal kinematic chain having a configuration of distal kinematic chain 300.

Referring now to FIG. 4, a non-limiting embodiment of a table-mounted manipulator system 4000 is illustrated. The system 4000 includes one or more manipulators 415 (four manipulators 415_1 through 415_4 shown) that can include a distal kinematic chain such as kinematic chain 100, 200, 300, or 500 described above or as will be described in greater detail below. The system 4000 includes a table assembly 4001, at least one rail assembly 4002 coupled to the table assembly 4001 (two such rails are in the embodiment of FIG. 4 with only one visible in the view thereof), and one or more manipulators 415 coupled to each rail assembly 4002. Each manipulator 415 may carry one or more instruments (not shown), which may be removably or permanently mounted thereon. The system 4000 also may include a control system (not illustrated) to control operations thereof and a user input and feedback system (not illustrated) to allow a user to provide inputs to operate the system 4000.

In some embodiments, the system 4000 is configured as a computer-assisted, teleoperable medical system, in which case table assembly 4001 may be configured to support a patient (not shown) or other workpiece and the manipulators may be coupled to medical instruments. The system 4000 in this configuration may be usable, for example, to perform any of a variety of medical procedures, such as surgical procedures, diagnostic procedures, imaging procedures, therapeutic procedures, etc. Moreover, the system 4000 when configured as a teleoperable medical system need not necessarily be used on a living human patient. For example, a non-human animal, a cadaver, tissue-like materials, or other workpiece used for training purposes, and so on, may be supported on the table assembly 4001 and worked on by system 4000. In other embodiments, the system 4000 is configured as a computer-assisted teleoperable system for use in non-medical contexts, in which case the table assembly 4001 may be configured to support an inanimate workpiece (something being manufactured, repaired, tested, etc.) and the instruments may be non-medical instruments, such as industrial instruments.

As shown in FIG. 4, the table assembly 4001 includes a platform assembly 4003 configured to support the patient or inanimate workpiece, a support column 4004 coupled to and supporting the platform assembly 4003, and a base (not illustrated) coupled to the support column 4004. One or more manipulators 415 are movably coupled to the rail 4002 such that the manipulators 415 can translate along the rail 4002. A base may be configured to contact the ground or other surface upon which the table assembly 4001 rests to provide stability for the table assembly 4001. In some embodiments, the base includes mobility features, such as wheels, tracks, or other such features, to allow movement of the table assembly 4001 along the ground or other surface.

In some embodiments, various of the above-described parts of the table assembly 4001 may be movable relative to one another. For example, in some embodiments the platform assembly 4003 as a whole can be moved relative to the support column 4004, such as by tilting around a horizontal axis, swiveling around a vertical axis, translating vertically along the support column 4004, translating horizontally relative to the support column 4004, and so on. In some embodiments, the platform assembly 4003 may have multiple sections that are rotatable relative to each other so as to provide differing inclinations along a surface of the platform assembly 4003 that supports the workpiece.

As noted above, the system 4000 also includes one or more manipulators 415. FIG. 4 illustrates four manipulators 415_1, 415_2, 415_3, 415_4, with two positioned along each longitudinal side of the platform assembly 4003, but any number of manipulators 415 may be included (such as, for example, one, two, three, or more manipulators mounted to each rail assembly 4002). A manipulator 415 may include a kinematic chain of links coupled together by one or more joints, including a distal kinematic chain 400 (for simplification only labeled on manipulator 415_3) including a distal link 410 and wrist mechanism 420 in accordance with the various embodiments described herein. The kinematic chain 300 may be used as the distal kinematic chain 400 for each of the manipulators 415, although different manipulators 415 can optionally have different configurations. As such the manipulators 415 further include a spar 450, carriage 455, and a canula mount 460 (again for simplification only labeled on manipulator 415_3). As illustrated in FIG. 4, the manipulators 415 each further include a proximal link assembly including a plurality of proximal links connected via joints between the rail 4002 and the distal link 410 of the distal kinematic chain 400. The arrangement of the links and joints of the proximal link assembly of the manipulator system 4000 in FIG. 4 are exemplary and not limiting of other arrangements that are contemplated as within the scope of the disclosure.

The manipulators 415 are movable through various degrees of freedom of motion provided by various joints, including the movements of the distal kinematic chain 400 as described above with reference to the degrees of freedom discussed for the distal kinematic chain 300. This allows an instrument mounted to the manipulator 415 to be moved relative to the worksite. Some of the joints may provide for rotation of links relative to one another, other joints may provide for translation of links relative to one another, and some may provide for both rotation and translation, which can provide for various orientations of the manipulators 415, and the instruments (not illustrated) carried by the manipulators 415 relative to each other and the table assembly 4001.

While various embodiments described above and further below include a cannula mount as part of the distal kinematic chain of a manipulator, embodiments without such a cannula mount are also envisioned, in which case the cannula mount can be excluded or a different mount may be used to directly hold a portion of the instrument coupled to the carriage of the spar.

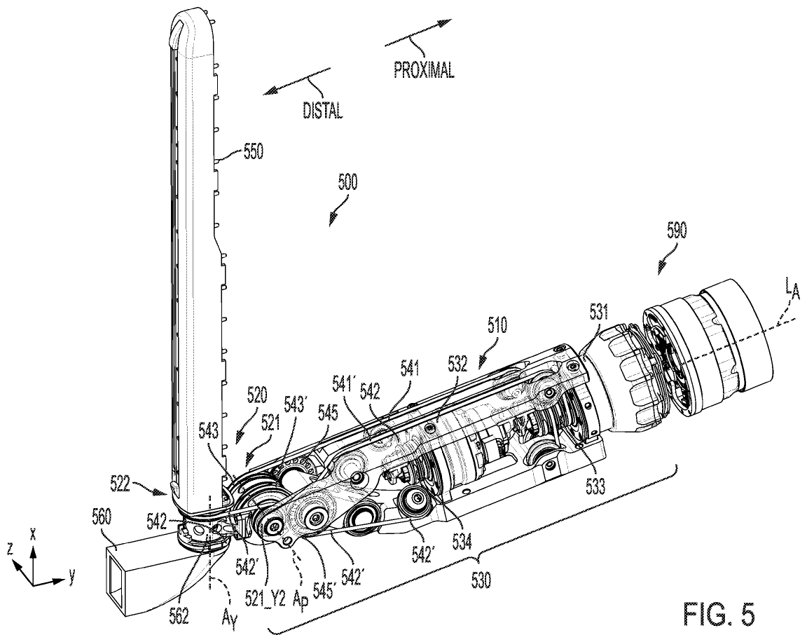

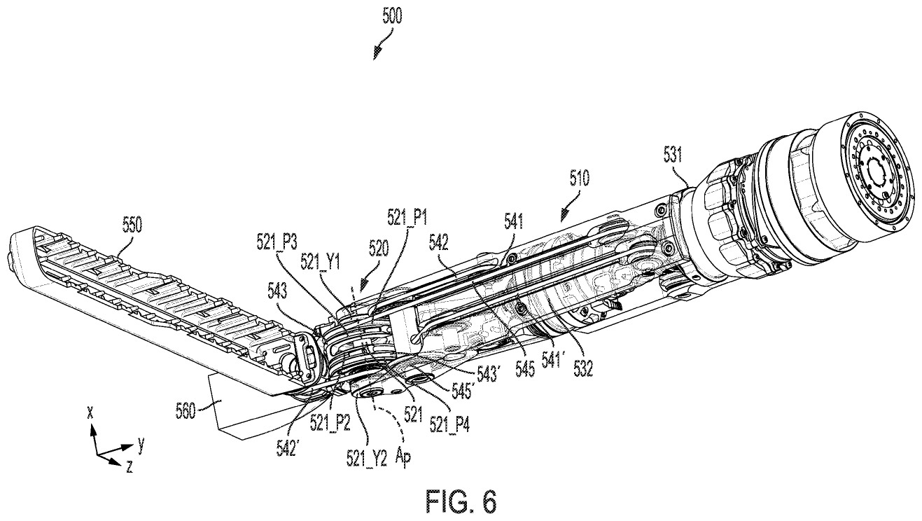

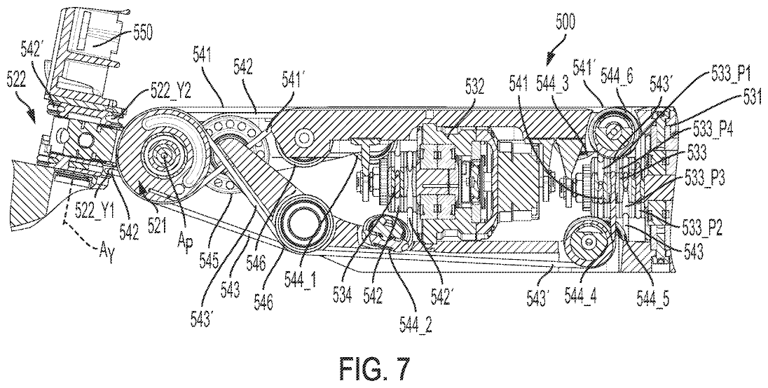

With reference now to FIGS. 5-10, various views of another embodiment of a distal kinematic chain and drive system that can be used in a manipulator configured to support and actuate an instrument, such as, for example, the manipulator 300 or 400, are depicted. FIGS. 5 and 6 are side and top perspective views of a kinematic chain 500 and drive system, while FIGS. 7-10 are various partial side views of the kinematic chain 500 and drive system.

As with other embodiments described above, the embodiment of FIGS. 5-10 includes a kinematic chain 500 including a distal link 510 (with outer housing components of the link not shown in the various views for ease of illustration), a wrist mechanism 520 that provides both yaw and pitch articulation to a spar 550 supported by the wrist mechanism 520. A carriage (not illustrated in FIGS. 5-9) configured to support and transmit actuation force to an instrument (not illustrated in FIGS. 5-10) can be movably coupled to the spar 550 so as to be able to translate along the spar 550 (as described above with reference to FIGS. 3 and 4 for example). A cannula mount 560 can be further coupled to the spar 550 and configured to hold a cannula (not illustrated) so that a distal portion of an instrument held by the carriage can be advanced or withdrawn through the cannula via translation of the instrument carriage along the spar 550. As discussed above, the cannula mount and cannula are optional components.

The proximal end portion of distal link 510 (with proximal and distal directions labeled by the arrows in FIG. 5) can be further coupled to a proximal rotational joint 590 that allows the distal link 510 with the wrist mechanism 520, spar 550, and cannula mount 560 (if any) to roll about a longitudinal axis L_{A} of the distal link 510. As with various other embodiments described above, the wrist mechanism 520 can include a pitch articulable joint structure and a yaw articulatable joint structure, with a respective pitch axis A_{P} and yaw axis A_{Y} of rotation being shown.

The drive system of the embodiment of FIGS. 5-10 that is operably coupled to articulate the joint structures of the wrist mechanism 520 can have similar components and operate in a manner similar to that described above with reference to the drive system 130, 230 of FIGS. 1 and 2. The drive system 530 of the embodiment of FIGS. 5-10 includes drive devices 531, 532 disposed in relative distal and proximal locations to each other along a length of the distal link 510 and located remotely from the joint structures toward a proximal end portion of the link 510. The drive devices 531, 532 are motors having drive shafts with capstans 533, 534 coupled to shafts. The motors 531, 532 are arranged such that the rotational axes of the drive shafts and thus the capstans 533, 534 are generally oriented approximately parallel to the longitudinal axis L_{A} and perpendicular to the axis A_{P}. A third drive device (e.g., third motor) can be operably coupled to drive the rotation joint 590 and thus roll DOF of the link 510.

The joint structures 521, 522 include pulley mechanisms, as will be further described below, with actuation members in the form of tension members, such as cables or similar members, coupled to the pulley mechanisms to transmit forces. The actuation members are further coupled at opposite ends to the capstans 533, 534.

For example, in a manner similar to the drive systems 130, 230 of the embodiments of FIGS. 1 and 2 described above, to transmit forces from the drive devices 531, 532 to the joint structures 521, 522 of the wrist mechanism 520, actuation members in the form of tension members, such as cables or similar members, can be used. As shown, two pair of cables 541, 541′, 543, 543′ are coupled between the drive device 531 and a pulley mechanism of the joint structure 521 and one pair of cables 542, 542′ is coupled between the drive device 532 and a pulley mechanism of the joint structure 522. The drive device 531 can transmit force to respectively pay in and pay out the pairs of cables 541, 543 and 541′, 543′ to articulate the joint structure 521 in opposite directions about the axis A_{P}. The drive device 532 can transmit force to respectively pay in and pay out the pairs of cables 542 and 542′ to articulate the joint structure 522 in opposite directions about the axis A_{Y}. The cables 541, 541′, 543, 543′ can extend past and be positioned laterally of the second drive device 532 between the pulley mechanism of joint structure 521 and the drive device 531.

While two pairs of cables are shown operably coupling the drive device 531 and joint structure 521, one pair or more than two pairs could be used. While one pair of cables is shown operably coupling the drive device 532 and joint structure 522, two or more pairs could be used. Each additional pair of cables operably coupling a drive device and a joint structure can be redundant to a single pair. The number of pairs of cables may be chosen depending on the overall loads the cables may be subjected to. For example, in the embodiment of FIGS. 5-10, the pitch articulation joint structure may have a large range of motion and be subject to greater overall loads and thus two pairs of cables 541, 541′ and 543, 543′ are used. In view of such load and desired articulation, the motor of drive device 531 also can be larger than that of drive device 532. Further, in alternative embodiments, a pair of cables can be replaced with a single cable that loops around either the associated pulley or capstan.

As with various embodiments described above, the drive system 530 employs cable routing arrangements around the pulley mechanisms of the joint structures 521, 522 and the capstans 533, 534 that conserve space, while maintaining desirable joint stiffness and other desirable dynamics. As can be seen in the various views of FIGS. 5-10, the joint structure 521 includes a pulley mechanism that includes four pulley surface grooves 521_P1, 521_P2, 521_P3, 521_P4 that receive the pairs of cables 541, 541′ 543, 543′ coupled to the capstan 533 of drive device 531 in the embodiment of FIG. 1. The pair of cables 541, 541′ are respectively received in a pulley surface grooves 521_P1 and 521_P2 and wrap in opposite directions about the pulley mechanism, thereby being paid in/paid out oppositely in response to rotation of the capstan 533. The pair of cables 543, 543′ are respectively received in pulley surface grooves 521_P3 and 521_P4, and also wrap in opposite directions about the pulley mechanism, thereby being paid in/paid out oppositely in response to rotation of the capstan 533.

The joint structure 522 includes a pulley mechanism with two pulley surface grooves 522_Y1 and 522_Y2 to respectively receive the pair of cables 542, 542′, which operably couple the capstan 534 of drive device 532 and the pulley mechanism 522. The pair of cables 542, 542′ wrap in opposite directions about the pulley mechanism the pulley mechanism 522, enabling the pair of cables 542, 542′ to pay in and pay out oppositely in response to rotation of the pulley mechanism 522. Further, the pair of cables 542, 542′ wrap in opposite directions around the pulley mechanism of joint structure 521 as they extend from the joint structure 522 to the capstan 534. More specifically a pair of outer pulley surface grooves 521_Y1, 521_Y1 are disposed on opposite sides of the series of grooves 521_P1-521_P4 and respectively receive the cables 542, 542′ as they extend from the pulley mechanism of joint structure 522 to the capstan 534.

As described above, by routing the cables 542, 542′ around the pulley mechanism of joint structure 521, again in oppositely wrapped directions, as well as coupling the cables 542, 542′ to the pulley mechanism of joint structure 522, length conservation of the cables 542, 542′ can be maintained when the pulley mechanism of joint structure 521 is actuated by the drive mechanism 531 by relative paying in and paying out of the cables 541, 541′, 543, 543′. As shown in FIG. 5, the pulley surface grooves 521_Y1, 521_Y2 may be around a smaller circumferential surface than those of pulley surface grooves 521_P1, 521_P2. Thus, the cables 542, 542′ pay in and pay out to a lesser extent for the rotation of the pulley mechanism of pitch articulation joint structure 521. While the present disclosure contemplates the pulley surface grooves 521_Y1, 521_Y2 may have a smaller circumference than the pulley surface grooves 521_P1, 521_P2, they could be the same or larger than the pulley surface grooves 521_P1, 521_P2. However, this can increase the bending stress of the cables. Further, it can result in an increase in the axial length of the joint 522.

The manner in which the cables of 542, 542′ result in the coupling of yaw articulation to pitch articulation in the absence of driving the capstan 534 to counteract the paying in and paying out of cables 542, 542′ that would occur otherwise, as well as the coupling of the braking of the drive device 532 to control braking of the yaw articulation of joint structure 522, are similar to that described above with reference to FIG. 2 and is not described again here to avoid redundancy.

In various embodiments, redundant cables can be routed such that they begin and end different planes horizontal relative to the longitudinal axis of the link, as perhaps best shown in FIG. 7. In other words, the cable 541′ starts at a bottom portion of pulley 521 and the link 510 and is routed up toward a top of the link 510 and capstan 533, and vice versa for the cable 543′. This can allow for more compact axial packaging of idler pulleys used to guide the cables to a capstan, described further below. In addition, such routing allows the pull force in each direction to be balanced for the pair of cables pulling in the same articulation direction of a joint, e.g., joint structure 521. This in turn can result in there being effectively no moment load on the output shaft of the motor driving the redundant pairs of cables (e.g., motor 531 in the embodiment of FIGS. 5-10). The present disclosure, however, contemplates other routing arrangements, including the redundant cables being routed along the top only or the bottom only, or other routing paths; the routing arrangement shown in the embodiment of FIGS. 5-10 is nonlimiting.

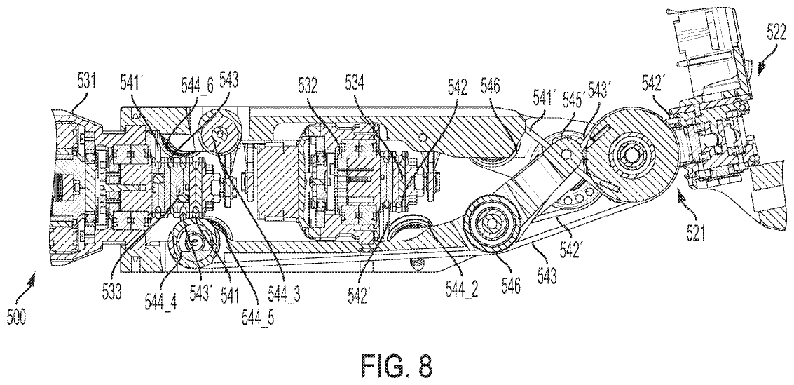

The capstans 533, 534 also have surface grooves to receive the different respective cables wrapping around the capstans 533, 534. As best shown in the various views of FIGS. 5 and 7-10, capstan 533 has four grooves 533_P1, 533_P2, 533_P3, and 533_P4, respectively receiving cables 541, 541′, 543, and 543′. Capstan 534 has two grooves 534_Y1, 534_Y2 respectively receiving cables 542, 542′. Optionally, a system of idler pulleys can also be used as needed along the routes of the cables 541, 541′ 542, 542′, 543, 543′ to alter the direction of the cables 541, 541′ 542, 542′, 543, 543′ and orient fleet angles of the cables 541, 541′, 542, 542′, 543, 543′ to enter the various grooves of the capstans 533, 534 so that the cables 541, 541′ 542, 542′, 543, 543′ enter grooves in a generally tangential manner to the outer circumferential surface of the capstans 533, 534. With reference to FIGS. 7 and 8, idler pulleys 544_1 through 544_6 are shown and respectively guide and orient the various cables 541, 541′, 542, 542′, 543, 543′ to a desired fleet angle introduction to the grooves 533_P1-533_P4 and 534_Y1-534_Y2 of the capstans 533 and 534. Additional idler pulleys 546 are situated between the pulley mechanism of joint structure 521 and the capstan 534 to assist with guiding and maintaining tension in the cables 541, 541′, 543, 543′ as they extend toward the more proximally located capstan 533.

The embodiment of FIGS. 5-10 also optionally includes an additional cable routing mechanism that allows for a relatively large range of pitch articulation of joint structure 521 without a risk of the cables 542, 542′ that couple to actuate the joint structure 522 and are guided over the pulley mechanism of the pitch joint structure 521 becoming unwrapped (or otherwise running out of cable length) relative to the pulley mechanism of joint structure 521. As depicted in FIGS. 5-10, to allow for a larger range of pitch articulation. As illustrated in FIGS. 5-10, just proximal to the pulley surface grooves 521_Y1, 521_Y2 from which the cables 542, 542′ wrap over, are a pair of idler pulleys 545, 545′ respectively receiving the cable 524, 542′. In the embodiment of FIGS. 5-10, the idler pulleys 545, 545′ are arranged such that their axes of rotation are offset from each other and from the pitch axis of rotation A_{P}.

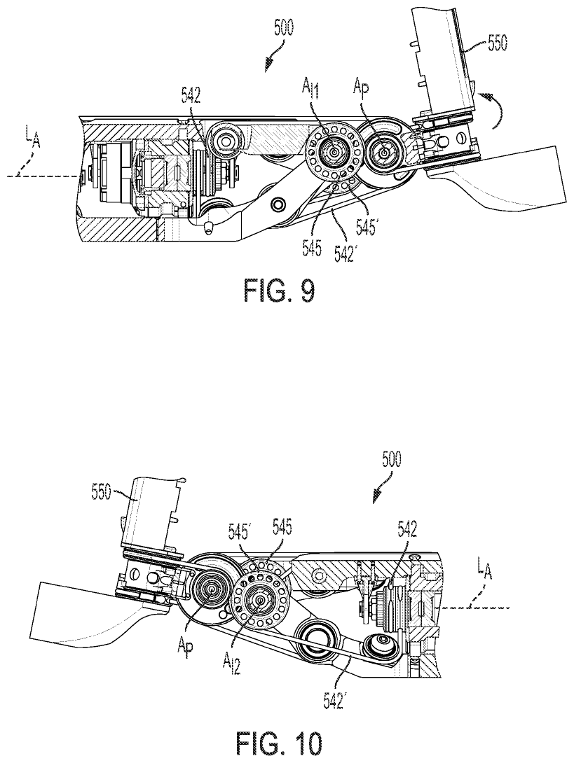

More specifically, as perhaps best shown in FIGS. 9 and 10 which are side views of a distal portion of the distal kinematic chain 500 and drive systems of the embodiment of FIGS. 5-10, the axis of rotation A_{I1} of idler pulley 545 over which the cable 542 wraps is slightly above or higher than the pitch axis of rotation A_{P} (i.e., along the direction perpendicular to the longitudinal axis L_{A} and the pitch axis A_{P}; in other words, the vertical direction in the orientation of FIGS. 9 and 10), and an axis of rotation A_{I2} of the idler pulley 545′ over which cable 542′ wraps is slightly under or lower than the pitch axis of rotation A_{P}. The offsets can roughly maintain the radial distance from A_{P} to A_{I2} and can be axi-symmetric in various embodiments; in other words, the same distance relative to the axis A_{P} but in opposite directions.

This offset arrangement of the idler pulleys 545, 545′ allows for an overall range of pitch articulation of greater than +/−80 degrees (greater than a total range of articulation of 160 degrees). Moreover, by offsetting the axis A_{I1} higher, the range of pitch back motion of the spar 550 as shown by arrow P_{B} in FIGS. 9 and 10 can be greater than in the opposite direction of pitch. For example, the pitch back range of motion can be up to 90 degrees. The articulation in the opposite direction can be about 70 degrees or more, for a total range of pitch articulation motion of 160 degrees or more. This can allow the spar 550 to rest almost parallel with or flush against the distal link 510, which can provide for more compact stowing configurations of a manipulator in some cases.

The offsets of the idler pulleys 545, 545′ can be selected as desired to achieve the desired ranges, including to utilizing no offsets. Thus, in some embodiments, it is contemplated that the offsets are not equal or are reversed, with A_{I1} being lower than A_{I2}. Further, it is contemplated as within the scope of the present disclosure that various embodiment utilize an arrangement of idler pulleys 545, 545′ having axes A_{I1}, A_{I2} that are not offset from the pitch axis of rotation A_{P}.

The present disclosure further contemplates a capstan arrangement that can provide relatively robust joint operation and stiffness while allowing for an effective paying in and paying out of cables from the capstan with relatively small dimensions for the capstan. In general, as a joint size increases, the amount of cable that needs to pay in and out from the capstan increases, and thus typically so does the capstan size. Moreover, increasing the diameter of the capstan, moves the idler pulleys that guide cables onto the capstan further radially outwardly, thus further increasing the overall dimensions required for the distal link. While larger joint diameters can provide desirable stiffness, smaller joint diameters can be desirable for moving the joint closer to the remote center of motion and for reducing mass. For smaller joint sizes, redundant or parallel cables can be used to maintain joint stiffness. With a single cable or cable pair, the cable diameter would need to get larger to offset the reduced stiffness of a smaller joint. Having a larger cable can also result in an increased diameter of the associated idler pulleys and/or the capstan to maintain the same cable bending stress, resulting in the overall link size growing in order to achieve a smaller joint size.

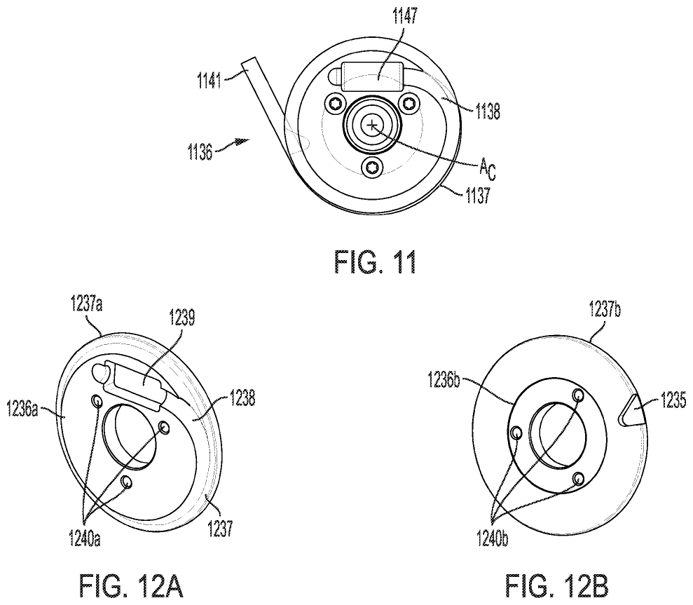

Capstans in accordance with various embodiments can allow for an effective wrap of cable of about 270 degrees while enabling a relatively small axial length. With reference now to FIG. 11, an embodiment of a capstan module that can be used to capture and route a single cable in accordance with the present disclosure is depicted. The capstan module 1136 captures the corresponding cable 1141 in an interior region of the capstan module 1136 between the axis of rotation A_{C} and an outer peripheral surface 1137 of the capstan module 1136 over which the cable 1141 wraps. From the location at which the captured end of the cable 1141 extends from the interior region of the capstan module 1136, the cable 1141 is guided along a modified spiral path 1138 to the outer peripheral surface 1137, which may have a groove along which the cable 1141 can be received as it pays in and pays out relative to the capstan 1136. This arrangement allows the overall path of the cable 1141 to remain in the same plane as it spirals out toward the outer peripheral surface 1137, and thus also uses a relatively smaller axial dimension for the capstan module 1136. As also shown in FIG. 11, to retain the cable 1141, an end portion of the cable 1141 can have a crimp 1139 that is captured by the capstan module 1136, although other securing mechanisms can be utilized.

With reference to FIGS. 12A and 12B, one embodiment of a structure for a capstan module, such as, for example, capstan module 1136, is a two-piece construction that includes two annular plates 1236a and 1236b, shown respectively in FIGS. 12A and 12B. Annular plate 12A has an inner surface that defines a crimp pocket 1239 and recessed path 1238 leading from the crimp pocket 1239 to an outer periphery 1237 of the inner surface portion of the annular plate 1236a. A stepped flange 1237a surrounds the inner surface region and defines an outermost peripheral circumference of the plate 1236a. The crimp pocket 1239 is configured to receive the crimp, such as crimp 1147, at an end portion of a cable, to assist in retaining the cable relative to the plate 1236a. The recessed path 1238 guides the cable from the crimp pocket 1238 in a modified spiral configuration toward the outer peripheral surface 1237 over which the cable wraps.

The other annular plate 1236b, shown in FIG. 12B, has a generally flat surface and peripheral circumference 1237b having outer diameter that matches the diameter of peripheral circumference 1237a. The two plates 1236a, 1236b are configured to be secured together, such as via connectors (not shown) through corresponding connector holes 1240a, 1240b in each plate. In the secured together state, the annular plates 1236a, 1236b define a groove defined by the outer peripheral surface 1237 the flange 1237a, and the portion of the plate 1236b that is radially outward of the peripheral surface 1237 and extends to the outer peripheral surface 1237b. The groove is configured to receive the cable from the recessed path 1238 and wrap the cable around the capstan module.

In another embodiment, not shown, the annular plate 1236b could have a mirrored configuration to that of annular plate 1236a, so that the overall recesses for capturing the crimp and cable is formed between the two plates.

One or more of capstan modules can be arranged along a common axis of rotation, such as a motor shaft, so as to provide an overall capstan to pay in and pay out one or more corresponding cables. For example, the embodiment of FIGS. 5-10 may utilize a capstan arrangement comprising the capstan module embodiments of FIGS. 11-12, with two such capstan modules being used together for capstan 534 and four such capstan modules being used together for capstan 533. In other words, a number of capstan modules of FIGS. 11 and 12 can be on a common drive shaft and driven together, with each receiving a corresponding cable for which a common paying in and paying out is desired and can be achieved via rotation of the capstan modules together on the common drive shaft.

While the capstan modules of the embodiments of FIGS. 11-12 can be utilized for the capstans of the embodiment of FIGS. 5-10, the embodiments of FIGS. 5-10 is not limited to such a capstan module arrangement and other capstan configurations may be utilized without departing from the scope of the present disclosure.

In another embodiment, the capstan module arrangement of FIGS. 11 and 12 can assist with tensioning of the cables. The capstan modules can be provided with features that permit insertion of a rack and pinion tightening tool that can be utilized to rotate each capstan/cable module individually and in situ when assembled together along the motor shaft.

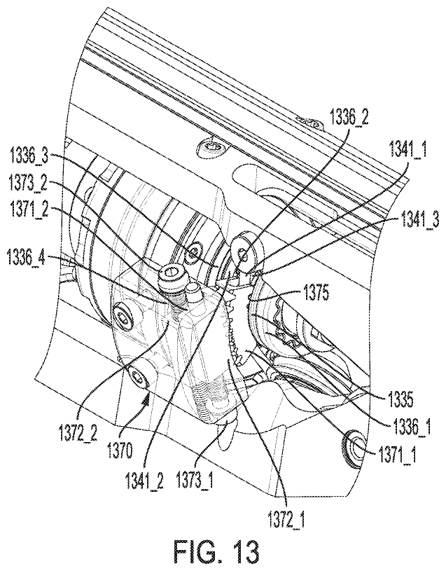

FIG. 13 depicts an embodiment of a rack and pinion tightening tool 1370 that can be utilized to tighten the individual capstan modules of a capstan. In the embodiment, a plurality of capstan modules 1336_1-1336_4, having a structure like discussed above with reference to FIGS. 11 and 12, are each strung on a common drive shaft of a motor 1331, but due to their individual nature can be separately rotated around that shaft to tension the respective cables wrapped around each individual capstan module 1336, with cables 1341_1-1341-3 visible in FIG. 13, wrapped.

The rack and pinion tightening tool 1370 comprises one or more driven toothed members 1371 configured to be engaged respectively with the capstan modules 1136_1-1336_4, and one or more corresponding driving toothed members 1372 having complementary teeth configured to mesh with the teeth on the one or more driven toothed members 1371. The driving toothed members 1372 are operably coupled to lead screws 1373_1, 1373_2 of the rack and pinion system 1370. Driving the lead screws 1373_1, 1373_2 can move the driving toothed members 1372 relative to the driven tooth members 1371 to thereby cause the driving toothed members 1372 to rotate the capstan modules 1336 and tighten (or loosen) the cables 1341. The rack and pinion tightening tool can be fixedly coupled to a fixed location in the vicinity of the capstan modules 1336 as they are located in situ within a kinematic chain. In the embodiment of FIG. 13, the tool 1370 can be configured to be removably connected at connection locations provided in a flange of the motor adjacent to where the capstan modules 1336 are positioned on the motor shaft.

As depicted in FIG. 13, the lead screws 1373_1, 1373_2 can drive the corresponding driving toothed members 1372_1, 1372_2 in opposite directions, which thereby drives the driven toothed members 1371_1, 1371_2 in opposite directions so as to account for differing directions of cable wrapping on the respective capstan modules 1336_1, 1336_2.

To engage the driven toothed members 1371 with the capstan modules 1336, the capstan modules can in various embodiments be provided with surface features that engage a complementary surface feature of the driven toothed members 1371. For example, referencing FIG. 12B, at least one of the plates (1236b shown) can include a surface feature 1135 in the form of a recess configured to engage a complementary surface feature 1375 in the form of a protrusion on the driven toothed member 1371. The embodiment of FIG. 13 shows the capstan module 1336_1 provided with a similar surface feature 1335 and the engagement of a corresponding protruding surface feature 1375 of the driven toothed member 1371_1. The configuration of the complementary surface features 1335, 1375 can be selected to provide a resistant fit to allow the driven toothed member 1371 to tighten and rotate the capstan module. It should be appreciated that other complementary surface features could be used to provide the desired removable engagement of the driven toothed members with the capstan modules. Other mechanisms to removably secure the driven toothed members 1371 to the capstan modules 1335 may be used aside from the use of the types of complementary surface features shown.

In other embodiments (not shown), the capstan module itself can have engagement features, such as teeth, a spur, a helical spiral, or similar to configured to engage and interact with corresponding features to provide a rack and pinion type of interaction with a tightening tool.

In various embodiments of a kinematic chain in accordance with any of the embodiments disclosed herein, it may be desirable to route other devices that provide functionality to be employed at a distal end of the kinematic chain, such as, for example, to an instrument supported at a distal end portion of the kinematic chain. By way of non-limiting example, it can be desirable to route various flux conduits from equipment that supplies flux located separately from a kinematic chain to locations at the distal end portion of the kinematic chain, such as to an instrument supported by, the kinematic chain. Flux conduits in medical applications can be conduits that are configured to supply an electrical current, pressure or gas (e.g. for insufflation or suction/evacuation), light, and other similar functionality which a medical instrument can use in the course of a medical procedure. Due to the numerous joints and degrees of freedom provided by the various embodiments of kinematic chain in accordance with the present disclosure, routing such flux conduits to the instrument can be relatively complex.

To address the issues with routing such flux conduits, the present disclosure contemplates a routing path for a flux conduit that includes a clock spring path about the proximally located joint structure of a wrist mechanism, from which the flux conduit feeds into a hollow region of the distal joint structure of the wrist mechanism via slots. Slots can be provided in distal joint structure 180 degrees circumferentially so that the flux conduit does not bend in response to the motions of that joint structure.

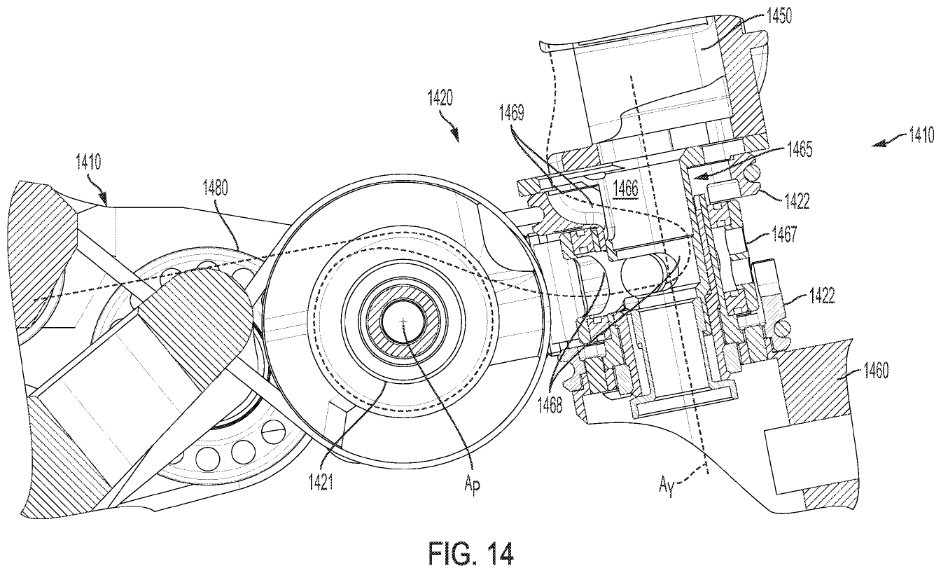

FIGS. 14-16 depict a cable routing arrangement for a flux conduit implemented for use with a distal kinematic chain, such as, for example, a distal kinematic chain 300, 400, or 500 that is part of a manipulator, though such a cable routing arrangement could also be used with the kinematic chains 100, 200 as well.

Referring now to FIG. 14, which shows a partial, sectional view of a distal end portion of a distal kinematic chain, the distal kinematic chain 1400 includes a distal link 1410 and a wrist mechanism 1420 supporting a spar 1450 and a cannula mount 1460 coupled to the spar 1450 (only a portion of the spar 1450 and cannula mount 1460 being depicted). Spar 1450 and cannula mount 1460 are coupled together via a common shaft portion 1465. A coupling 1467 secures to the shaft portion 1465 to the pulley mechanism of a joint structure 1422 configured to articulate relative to a yaw axis of rotation A_{Y}, which along with a joint structure 1421 that is configured to articulate relative to a pitch axis of rotation A_{P} of the wrist mechanism 1420. The coupling 1467 rotates with the rotation of the joint structure 1421, and the joint structure 1422 rotates within and relative to the coupling 1467, causing the spar 1450 and cannula mount 1460 to rotate about the axis A_{Y}.

The wrist mechanism 1420 can utilize the pulley-cable drive system arrangements discussed above with respect to various embodiments, such as for example, wrist mechanism 520 and the corresponding pulley and cable configurations used for the pitch articulation joint structure 521 and yaw articulation joint structure 522. The pulleys and cables are not further discussed here for simplification and to avoid redundancy.

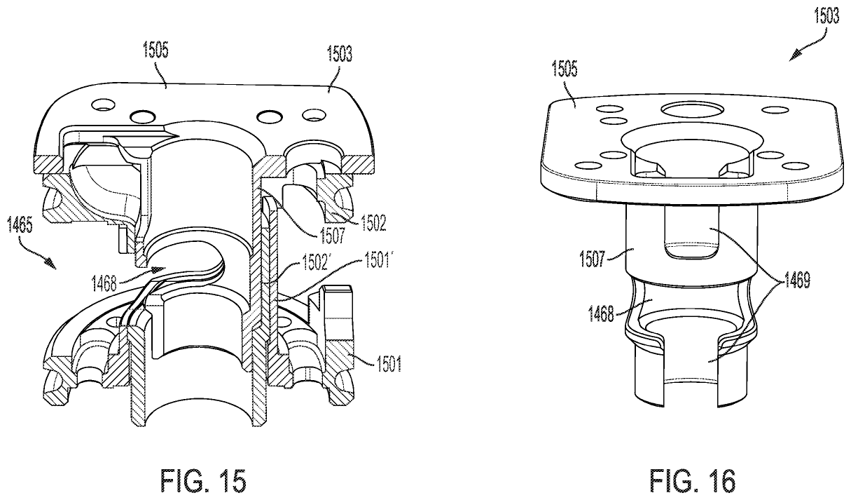

As illustrated in FIG. 14, with further reference to the isolated views of the overall shaft 1465 in the partial sectional view of FIG. 15 and the perspective view of the spar coupling member discussed further below in FIG. 16, the shaft 1465 and the coupling 1467 are hollow structures which provide an open space 1466 surrounding the yaw axis of rotation A_{Y}. Slots are provided through the coupling 1467 and shaft 1465. The slots 1468 are oriented circumferentially about axis A_{Y}, for example, over a range of 180 degrees circumferentially, so as to allow one or more cables (one cable 1480 depicted in dashed line) to be routed into the open space 1466 along a generally proximal to distal direction from the joint structure 1421 and with enough slack so that the cable 1480 does not bend significantly during yaw articulation of the joint structure 1422. With this arrangement, during articulation of the joint structure 1422 in yaw, the one or more cables 1480 will not be bent given the space to accommodate the cable(s) 1480, but can still twist on its own axis. More specifically, the slots 1468 can align to form an overall slot extending from the joint structure 1421 oriented generally orthogonally to the yaw axis A_{Y} and through which the one or more cables 1480 can pass to enter the open space 1466 in the joint structure 1422.

With reference now to FIG. 15, shaft 1465 is made up of three parts—the respective pulley portions 1501, 1502 of the pulley mechanism of joint 1422 and a spar coupling member 1503. The spar coupling member 1503 comprises a bracket portion 1505 that couples to the spar 1450 and a stem portion 1507 that couples to a shaft extension portion 1502′ of pulley portion 1502 to form shaft 1465, with a shaft extension portion 1501′ of pulley portion 1501 coupled to an surrounding shaft extension portion 1502′. As can be seen, cut outs are provided in each of these portions to form the overall slot 1468 of the shaft portion 1465

Referring now to FIGS. 16, slots 1469 are also provided in the stem portion 1507 of the spar coupling member 1503 that are oriented generally transverse to the orientation of the cutout portion of the stem portion 1507 that forms part of slot 1468. This arrangement of slots 1469 permits the one or more cables 1480 to be routed back proximally from within the open space 1466 of the shaft 1465 toward, but external to, the spar 1460, as illustrated in FIG. 14. This routing allows the spar 1460 to be connected and disconnected from the joint structure 1422 without pulling the one or more cables through a length of the spar 1460 or otherwise out of the wrist mechanism 1420.

As also illustrated in FIG. 14, the cable routing path can be a clockspring winding around the pitch axis A_{P} in the joint structure 1421. Utilizing a clockspring winding routing scheme can provide a relatively large single-sided bend radius.

Embodiments described herein may be well suited for use in any of a variety of medical procedures, as described above. Such procedures could be performed, for example, on human patients, animal patients, human cadavers, animal cadavers, and portions or human or animal anatomy. Medical procedures as contemplated herein include any of those described herein and include, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein may be used in, or may be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the da Vinci® Surgical Systems are illustrative and not to be considered as limiting the scope of the disclosure herein.

As used herein and in the claims, terms such as computer-assisted manipulator system, teleoperable manipulator system, or the like should be understood to refer broadly to any system comprising one or more controllable kinematic structures (“manipulators”) that are movable and controllable at least in part through the aid of an electronic controller (with or without human inputs). Such systems may occasionally be referred to in the art and in common usage as robotically assisted systems or robotic systems. Such systems include systems that are controlled by a user (for example through teleoperation), by a computer automatically (so-called autonomous control), or by some combination of these. In examples in which a user controls at least some of the operations of the manipulator, an electronic controller (e.g., a computer) may facilitate or assist in the operation. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems may be implemented in a variety of contexts to perform a variety of procedures, both medical and non-medical. Thus, although some examples described in greater detail herein may be focused on a medical context, the devices and principles described herein are also applicable to other contexts, such as industrial manipulator systems.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. In some instances, well-known circuits, structures, and techniques have not been shown or described in detail in order not to obscure the embodiments. Like numbers in two or more figures represent the same or similar elements.

Further, the terminology used herein to describe aspects of the invention, such as spatial and relational terms, is chosen to aid the reader in understanding example embodiments of the invention but is not intended to limit the invention. For example, spatial terms—such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

As used herein, “proximal” and “distal” are spatial/directional terms that describe locations or directions based on their relationship to the two ends of a kinematic chain. “Proximal” is associated with the end of the kinematic chain that is closer to the base or support of the chain, while “distal” is associated with the opposite end of the kinematic chain, which in the context of a manipulator may hold an instrument to be positioned and oriented. When used in to refer to locations or to portions of a component, proximal and distal indicate the relative positions of the locations or portions relative to the base of the chain, with the proximal location or potion being closer to the base (closer here referring to proximity along the kinematic chain, rather than absolute distance). When used to refer to a direction, “proximal” refers to directions that point generally from a given location along a kinematic chain towards a more proximal location along the kinematic chain, and “distal” refers to directions that point from the given location towards a more distal location along the kinematic chain.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least: any inconsequential variations; those variations that are typical in the relevant art for the type of item in question due to manufacturing or other tolerances; and/or variations that are within ±5% of the stated value, property, or relationship unless indicated otherwise.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A system for actuating an articulation mechanism, the system comprising:
a link extending along a longitudinal dimension from a proximal end portion of the link to a distal end portion of the link;
an articulation mechanism coupled to the distal end portion of the link, the articulation mechanism articulatable in pitch and yaw relative to the link;
a first motor housed at least partially in the link and operably coupled to transmit force to actuate the articulation mechanism to articulate in pitch relative to the link; and
a second motor housed at least partially in the link and operably coupled to transmit force to actuate the articulation mechanism to articulate in yaw relative to the link,
wherein the first motor and the second motor are arranged consecutively along the longitudinal dimension such that the first motor is closer to the proximal end portion of the link than the second motor.

2. The system of claim 1, wherein the second motor is distal to the first motor.

3. The system of claim 2, wherein the first motor is located at a proximal end portion of the link.

4. The system of claim 1, wherein the articulation mechanism comprises a first joint defining a pitch axis and a second joint defining a yaw axis.

5. The system of claim 4, wherein the first joint and second joint are offset from each other along the longitudinal dimension.

6. The system of claim 5, wherein the first joint is proximal the second joint.

7. The system of claim 4, wherein:
the first joint comprises a first cable-and-pulley assembly operably coupled to the first motor, and
the second joint comprises a second cable-and-pulley assembly operably coupled to the second motor.

8. The system of claim 7,
wherein the first cable-and-pulley assembly comprises a first pair of cables respectively operably coupled to a first pulley mechanism having a rotation axis along the pitch axis, the first pair of cables configured to be oppositely paid in and paid out in response to rotation of the first motor; and
wherein the second cable-and-pulley assembly comprises a second pair of cables respectively operably coupled to a second pulley mechanism having a rotation axes along the yaw axis, the second pair of cables configured to be oppositely paid in and paid out in response to rotation of the second motor.

9. The system of claim 8, wherein the first pair of cables extend past and laterally of the second motor between the first motor to the first pulley mechanism.

10. The system of claim 8, wherein the second pair of cables wrap on the first pulley mechanism in opposite directions.

11. The system of claim 8, further comprising:
a first capstan operably coupled to the first motor, the first pair of cables at least partially wrapping around the first capstan in opposite directions; and
a second capstan operably coupled to the second motor, the second pair of cables at least partially wrapping around the second capstan in opposite directions.

12. The system of claim 1, wherein the first motor and the second motor are configured to actuate the articulation mechanism independently in pitch and yaw.

13. The system of claim 1, further comprising one or more braking mechanisms configured to apply a braking force to brake one or both of the first motor and the second motor.

14. The system of claim 13, wherein the articulation mechanism is configured to be locked in pitch articulation in response to the one or more braking mechanisms applying the braking force to the first motor.

15. The system of claim 14, wherein the articulation mechanism is configured to be locked in yaw articulation in response to the one or more braking mechanisms applying the braking force to the first motor or wherein the articulation mechanism is configured to be locked in yaw articulation in response to the braking mechanism applying the braking force to the second motor.

16. The system of claim 1, wherein the link is a link of a manipulator arm of a teleoperated manipulator system, the manipulator arm comprising a manipulator drive interface configured to transmit actuation force to a medical instrument mounted to the manipulator drive interface.

17. The system of claim 16, wherein the manipulator arm further comprises a spar link coupled to the link via the articulation mechanism, the manipulator drive interface coupled to the spar link.

18. The system of claim 16, wherein the link is a distal link of the manipulator arm and is rotatably coupled to a proximal link of the manipulator arm.

19. The system of claim 18, wherein the distal link is configured to roll about a longitudinal axis of the distal link relative to the proximal link.

20. The system of claim 19, further comprising a third motor proximal the first motor and operably coupled to transmit force to actuate the distal link to roll about the longitudinal axis.

21-36. (canceled)
